SEQUENCE LISTING (2 sequences)

SEQ ID NO: 1 - DNA, 20 bp, Artificial Sequence
  Artificially synthesized oligonucleotide primer
ggaaccacctcaatgcaaag

SEQ ID NO: 2 - DNA, 20 bp, Artificial Sequence
  Artificially synthesized oligonucleotide primer
aagcttctgtttggctgtcc

(19) United States
(12) Patent Application Publication
(10) Pub. No.: US 2012/0003225 A1
(43) Pub. Date: Jan. 5, 2012

(54) AUTOANTIBODIES IN THE DETECTION AND TREATMENT OF CANCER

(75) Inventors: Edward F. Patz, JR., Chapel Hill, NC (US); Michael J. Campa, Durham, NC (US); Elizabeth B. Gottlin, Chapel Hill, NC (US)

(73) Assignee: DUKE UNIVERSITY, Durham, NC (US)

(21) Appl. No.: 12/991,666

(22) PCT Filed: May 11, 2009
(86) PCT No.: PCT/US2009/043460

§ 371 Date: Sep. 16, 2011

Related U.S. Application Data
(60) Provisional application No. 61/127,138, filed on May 9, 2008.
(60) Provisional application No. 61/128,717, filed on May 23, 2008.
(60) Provisional application No. 61/188,209, filed on Aug. 7, 2008.

(51) Int. Cl. A61K 39/395; A61K 39/00; C07K 16/18; G01N 33/574

(52) U.S. Cl. 424/136.1; 436/501; 424/174.1; 424/277.1; 424/158.1; 424/198.1; 530/389.2; 530/389.1

(57) ABSTRACT

Methods, kits, and compositions for detecting and/or treating cancer based on detecting and/or administering an antibody, which can optionally be a bispecific antibody, having the immunoreaction characteristics of a cancer-associated autoantibody; an antigen for a cancer-associated autoantibody; or both an antibody having the immunoreaction characteristics of a cancer-associated autoantibody and an antigen for a cancer-associated autoantibody, in or to a subject in need thereof.

### RELATED APPLICATIONS

The presently disclosed subject matter claims the benefit of U.S. Provisional Patent Application Ser. No. 61/127,138, filed May 9, 2008; U.S. Provisional Patent Application Ser. No. 61/128,717, filed May 23, 2008; and U.S. Provisional Patent Application Ser. No. 61/188,209, filed Aug. 7, 2008, the disclosures of which are incorporated herein by reference in their entireties.

### GRANT STATEMENT

This study was partially supported by NCI grant 5RO1-CA109384-03 from the U.S. National Institutes of Health, National Cancer Institute. Thus, the U.S. government has certain rights in the presently disclosed subject matter.

### TECHNICAL FIELD

The presently disclosed subject matter pertains to the use of autoantibodies in the detection and treatment of cancer.

### BACKGROUND

Cancer continues to be a significant worldwide public health issue. More effective approaches for detecting and treating cancer continue to be pursued.

Taking the example of lung cancer, although advances in noninvasive imaging have improved the ability to detect lung cancer, >75% of lung cancer patients present with advanced stage disease when therapeutic options are limited. Mountain, C. F. Revisions in the International System for Staging Lung Cancer. Chest, 111: 1710-1717, 1997. Even those patients who present with clinical stage I lung cancer have at best a 60% 5-year survival rate, signifying that a large percentage of all stage I patients have undetectable metastatic disease at the time of presentation. Mountain, C. F. Revisions in the International System for Staging Lung Cancer. Chest, 111: 1710-1717, 1997. These statistics underscore the need for improvements in early detection strategies.

Additionally, lung cancer accounts for more cancer deaths than any other malignancy. Despite advances in diagnostic capabilities and treatment, lung cancer mortality has not significantly changed over the past several decades. Most patients present with inoperable disease when therapeutic options including chemotherapy and radiotherapy are rarely curative.

Accordingly, there remains an unmet need for approaches that provide for the detection and treatment of cancer, including but not limited to lung cancer.

### SUMMARY

The presently disclosed subject matter pertains at least in part to methods for detecting cancer in a subject. In some embodiments, the method can comprise detecting the presence and/or amount of a cancer-associated autoantibody in a sample from the subject.

A method for managing treatment of a subject with potential cancer is also disclosed. In some embodiments, the method comprises detecting the presence and/or amount of a cancer-associated autoantibody in a sample from the subject; and managing the treatment of the subject with potential cancer based on the presence or amount of the cancer-associated autoantibody.

A method for molecular staging of a tumor or suspected tumor is also disclosed. In some embodiments, the method comprises detecting the presence and/or amount of a cancer-associated autoantibody in a sample from the subject; and determining the molecular stage of the tumor or suspected tumor based on the presence or amount of the cancer-associated autoantibody.

A method for assigning a subject to a high-risk group for cancer is also disclosed. In some embodiments the method comprises detecting the presence and/or amount of a cancer-associated autoantibody in a sample from the subject; and assigning the subject to a group having a high-risk of cancer based on the presence or amount of the cancer-associated autoantibody.

In the presently disclosed methods the sample can be a serum sample or a blood sample. In the presently disclosed methods the subject can be a human subject.

In some embodiments the cancer is lung cancer. In some embodiments, the methods can comprise detecting an autoantibody against Complement Factor H (CFH), an autoantibody against alpha-glucosidase (GANAB), an autoantibody against STIP1 (Stress-induced-phosphoprotein 1), an autoantibody against alpha-enolase, an autoantibody against 14-3-3 (14-3-3 protein epsilon), and/or an autoantibody against HSP 60 (60 kDa heat shock protein). In some embodiments, the methods can comprise detecting an autoantibody against one or more of the entities listed in Table 4, or any combination thereof.

The presently disclosed subject matter also provides in some embodiments a kit for detecting the presence and/or amount of a cancer-associated autoantibody in a sample from the subject. In some embodiments, the kit can comprise a binding partner specific for a cancer-associated autoantibody; and directions for detecting the presence of and/or measuring the amount of a cancer-associated autoantibody in a sample from the subject. In some embodiments the binding partner can be a binding partner specific for an autoantibody against Complement Factor H (CFH), a binding partner specific for an autoantibody against alpha-glucosidase (GANAB), a binding partner specific for an autoantibody against STIP1 (Stress-induced-phosphoprotein 1), a binding partner specific for an autoantibody against alpha-enolase, a binding partner specific for an autoantibody against 14-3-3 (14-3-3 protein epsilon), or a binding partner specific for an autoantibody against HSP 60 (60 kDa heat shock protein). In some embodiments, the binding partner can be a binding partner specific for an autoantibody against one or more of the entities listed in Table 4. In some embodiments any combination of the aforementioned binding partners is provided.

In some embodiments the binding partner is conjugated to a solid support, and the kit can comprise a second specific binding partner for the autoantibody. In some embodiments the second specific binding partner can be an antibody. In some embodiments, the second specific binding partner can be conjugated to a detectable group. The detectable group can be selected from the group including but not limited to radioactive labels, fluorescent labels, enzyme labels and fluorescent labels. The kit can comprise one or more of buffering agents, protein stabilizing agents, enzyme substrates, background reducing agents, control reagents, an apparatus for conducting the detection, and any necessary software for analysis and presentation of results.

The presently disclosed subject matter also provides in some embodiments a method of treating cancer in a subject. In some embodiments the method comprises administering to a subject having a cancer an effective amount of an antibody (which optionally can be a bispecific antibody) having the immunoreaction characteristics of a cancer-associated autoantibody, an antigen for a cancer-associated autoantibody, or both an antibody having the immunoreaction characteristics of a cancer-associated autoantibody and an antigen for a cancer-associated autoantibody.

In some embodiments, the cancer is lung cancer. In some embodiments the administering comprises administering an antibody against Complement Factor H (CFH), an antibody against alpha-glucosidase (GANAB), an antibody against STIP1 (Stress-induced-phosphoprotein 1), an antibody against alpha-enolase, an antibody against 14-3-3 (14-3-3 protein epsilon), or an antibody against HSP 60 (60 kDa heat shock protein). In some embodiments, the methods can comprise administering an antibody against one or more of the entities listed in Table 4. In some embodiments any combination of the aforementioned antibodies can be administered. In some embodiments the administering comprises administering a Complement Factor H (CFH) antigen, an alpha-glucosidase (GANAB) antigen, a STIP1 (Stress-induced-phosphoprotein 1) antigen, an alpha-enolase antigen, a 14-3-3 (14-3-3 protein epsilon) antigen, or a HSP 60 (60 kDa heat shock protein) antigen. In some embodiments, the methods can comprise administering an antigen prepared from one or more of the entities listed in Table 4. In some embodiments any combination of the aforementioned antigens can be administered. In some embodiments an adjuvant is administered to the subject.

A composition for treating cancer in a subject, the composition comprising an effective amount Of an antibody (which optionally can be a bispecific antibody) having the immunoreaction characteristics of a cancer-associated autoantibody, an antigen for a cancer-associated autoantibody, or both an antibody having the immunoreaction characteristics of a cancer-associated autoantibody and an antigen for a cancer-associated autoantibody; and a pharmaceutically acceptable carrier, is also provided in accordance with the presently disclosed subject matter. Optionally, the cancer is lung cancer.

In some embodiments the composition can comprise an antibody against Complement Factor H (CFH), an antibody against alpha-glucosidase (GANAB), an antibody against STIP1 (Stress-induced-phosphoprotein 1), an antibody against alpha-enolase, an antibody against 14-3-3 (14-3-3 protein epsilon), or an antibody against HSP 60 (60 kDa heat shock protein). In some embodiments, the composition can comprise an antibody against one or more of the entities listed in Table 4. In some embodiments the composition can comprise any combination of the aforementioned antibodies. In some embodiments the composition can comprise a Complement Factor H (CFH) antigen, an alpha-glucosidase (GANAB) antigen, a STIP1 (Stress-induced-phosphoprotein 1) antigen, an alpha-enolase antigen, a 14-3-3 (14-3-3 protein epsilon) antigen, or a HSP 60 (60 kDa heat shock protein) antigen. In some embodiments, the composition can comprise an antigen prepared from one or more of the entities listed in Table 4. In some embodiments the composition can comprise any combination of the aforementioned antigens. In some embodiments the composition can comprise an adjuvant.

The presently disclosed subject matter also provides in some embodiments an isolated and purified antibody having the immunoreaction characteristics of an autoantibody against Complement Factor H (CFH), an autoantibody against alpha-glucosidase (GANAB), an autoantibody against STIP1 (Stress-induced-phosphoprotein 1), an autoantibody against alpha-enolase, an autoantibody against 14-3-3 (14-3-3 protein epsilon), or an autoantibody against HSP 60 (60 kDa heat shock protein). The presently disclosed subject matter also provides in some embodiments an isolated and purified antibody having the immunoreaction characteristics of an autoantibody against one or more of the entities listed in Table 4.

Accordingly, it is an object of the presently disclosed subject matter to provide novel methods and compositions for detecting and treating cancer.

This and other objects are achieved in whole or in part by the presently disclosed subject matter.

An object of the presently disclosed subject matter having been stated above, other objects and advantages will become apparent upon a review of the following description, figures and examples.

### BRIEF DESCRIPTION OF THE DRAWINGS

FIGS. 1A and 1B are two immunoblots probed with NSCLC patient sera. FIG. 1A is a pooled serum blot: Ten individual serum samples from patients with Stage I NSCLC were used to probe a blot containing the pooled sera from 5 late stage NSCLC patients.

FIG. 1B is a CFH blot: Individual Stage I, Stage III/IV, and normal serum samples were used to probe a blot containing purified CFH.

FIGS. 2A, 2B and 2C depict expression, secretion, and binding of CFH to A549 vs. H661 cells.

FIG. 2A is a photograph showing RT-PCR of CFH RNA. cDNA was synthesized from RNA isolated from A549 or H661 cells and was amplified by RT-PCR with CFH-specific primers. The products were run in an agarose gel.

FIG. 2B is an immunoblot of secreted CFH. A549 and H661 cells were grown to 80% confluence in 75 cm² flasks. Concentrated conditioned media or 100 ng purified CFH were subjected to SDS-PAGE, blotted, and probed with a goat anti-human CFH primary antibody.

FIG. 2C is a bar graph presenting data from a CFH binding assay. Cells were incubated in triplicate with ¹²⁵I labeled CFH at 4° C. for 30 min. The cells were washed and bound cpm detected in a gamma counter. For competitive binding, 10 μg unlabeled CFH was added to the incubation 30 minutes before ¹²⁵I labeled CFH was added. CFH*=radiolabeled CFH. n.s.=not significant. Light bars: CFH*; dark bars: CFH*+CFH.

FIG. 3 is a bar graph showing deposition of C3 by lung cancer cells in the presence (+; dark bars) or absence (−; light bars) of CFH autoantibody; values are averages of triplicate measurements of each of three patients' IgG samples.

FIG. 4 is a photograph showing moderately differentiated lung adenocarcinoma demonstrates diffuse 3+ tumor cell immunostaining (magnified 200×) for CFH.

FIG. 5 is a Surf-Blot analysis for the detection of autoantibodies.

FIG. 6 depicts Coomassie staining of pooled adenocarcinoma cell lines lysate separated by 2D-PAGE. Circled spots were excised and sent for sequencing.

FIG. 7 is a Western blot of lysate against diluted serum from a lung adenocarcinoma patient. Signals were lined up with corresponding spots from FIG. 6.

FIGS. 8 and 9 are Surf-Blots of purified CFH probed with NSCLC patients' sera (FIG. 8, left) and matched controls (FIG. 9, right).

### DETAILED DESCRIPTION

In accordance with the presently disclosed subject matter, methods and compositions are provided for the detection, desirably early detection, of cancer in a subject. Methods and compositions are also provided for the treatment of cancer in a subject.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

### I. DEFINITIONS

While the following terms are believed to be well understood by one of ordinary skill in the art, the following definitions are set forth to facilitate explanation of the presently disclosed subject matter.

Following long-standing patent law convention, the terms “a” and “an” mean “one or more” when used in this application, including the claims.

Unless otherwise indicated, all numbers expressing quantities of ingredients, reaction conditions, and so forth used in the specification and claims are to be understood as being modified in all instances by the term “about”. Accordingly, unless indicated to the contrary, the numerical region of parameters set forth in this specification and attached claims are approximations that can vary depending upon the desired properties sought to be obtained by the presently disclosed subject matter.

“Amino acid sequence” and terms such as “peptide”, “polypeptide” and “protein” are used interchangeably herein, and are not meant to limit the amino acid sequence to the complete, native amino acid sequence (i.e. a sequence containing only those amino acids found in the protein as it occurs in nature) associated with the recited protein molecule. The proteins and protein fragments of the presently disclosed subject matter can be produced by recombinant approaches or can be isolated from a naturally occurring source. The protein fragments can be any size, and for example can range in size from four amino acid residues to the entire amino acid sequence minus one amino acid.

The term “antibody” includes whole antibodies as well as any antibody fragments or bispecific antibodies that bind with sufficient specificity to a protein or proteins of interest.

As used herein, the term “sample” is used in its broadest sense. In one sense, it is meant to include a specimen, from a biological source. Biological samples can be obtained from animals (including humans) and encompass fluids, solids, tissues, and gases. Biological samples include blood products, such as plasma, serum and the like.

As used herein, the term “subject” refers to any animal (e.g., a mammal), including, but not limited to, humans, non-human primates, rodents, and the like, which is to be the recipient of a particular treatment. The terms “subject” and “patient” are used interchangeably herein, such as but not limited to in reference to a human subject.

As used herein, the term “subject with potential cancer” refers to a subject that presents one or more symptoms indicative of cancer or is being screened for cancer (e.g., during a routine physical). A subject with potential cancer can also have one or more risk factors. A subject suspected of having cancer has generally not been tested for cancer. However, a “subject suspected of having cancer” can also encompass an individual who has received an initial diagnosis (e.g., a CT scan showing an indeterminate pulmonary nodule) but for whom the stage of cancer is not known. The term further includes people who once had cancer (e.g., an individual in remission).

As used herein, the term “subject at risk for cancer” refers to a subject with one or more risk factors for developing cancer.

### II. REPRESENTATION EMBODIMENTS

II.A. Detection Methods

The presently disclosed subject matter provides in some embodiments a way to detect cancer in a subject from a sample, including but not limited to a blood sample. In some embodiments, the presently disclosed subject matter can be carried out by looking for the presence of specific antibodies in the serum of subjects. When certain of these antibodies are present, cancer is likely. Individuals without cancer may not have these antibodies in their serum.

Thus, provided herein are methods for the detection of cancer, including but not limited to lung cancer, using the subject's own antibodies, referred to as “cancer-associated autoantibodies”, as the basis for a novel serum test. There are currently no serum tests for lung cancer, among other types of cancer. Other representative types of cancers include melanoma, adenocarcinoma, malignant glioma, prostatic carcinoma, kidney carcinoma, bladder carcinoma, pancreatic carcinoma, thyroid carcinoma, colon carcinoma, rectal carcinoma, brain carcinoma, liver carcinoma, breast carcinoma, ovary carcinoma, solid tumors, solid tumor metastases, angiofibromas, retrolental fibroplasia, hemangiomas, and Karposi's sarcoma. Additional exemplary cancers would be apparent to one of ordinary skill in the art upon a review of the instant disclosure.

In some embodiments, the presently disclosed subject matter directly addresses the problem of lung cancer detection. Since greater than three-fourths of subjects present with late-stage lung cancer, when treatment options are limited, early detection could potentially save tens of thousands of lives each year. Thus, the presently disclosed subject matter can be used in the following representative, non-limiting clinical scenarios: (1). determining individuals at high risk for cancer; (2) distinguishing which subjects with non-specific lesions on imaging studies have cancer from those who do not have cancer; (3) following subjects with cancer to predict prognosis.

Alone or in combination, the presently disclosed cancer-associated autoantibody markers provide significant clinical utility for the early detection of cancer (including but not limited to lung cancer) and the molecular staging of tumors. Representative autoantibody markers include but are not limited to autoantibodies against Complement Factor H (CFH), alpha-glucosidase (GANAB), STIP1 (Stress-induced-phosphoprotein 1), alpha-enolase, 14-3-3 (14-3-3 protein epsilon), and HSP 60 (60 kDa heat shock protein). Additional representative autoantibody markers include but are not limited to autoantibodies respectively against the entities listed in Tables 2-4, presented herein below.

Also provided are methods for assigning a subject to a high-risk group for cancer, including but not limited to lung cancer. The method can comprise detecting the presence and/or amount of a cancer-associated autoantibody in a sample from the subject and determining whether the subject should be assigned to the group having a high-risk of lung cancer based on the presence and/or amount of a cancer-associated autoantibody in the sample from the subject.

In some embodiments, a method is provided for managing treatment of a subject with cancer or with potential cancer. The method can comprise detecting the presence and/or amount of a cancer-associated autoantibody in a sample from the subject; and managing the treatment of the subject with cancer or potential cancer based on the presence and/or amount of a cancer-associated autoantibody in a sample from the subject.

The presence or level of the presently disclosed autoantibodies is determined in a variety of animal tissues. In some embodiments, the autoantibodies are detected in animal tissue or bodily fluids. In some embodiments, the autoantibodies are detected in bodily fluids including plasma, serum, whole blood, mucus, and/or urine. In a particular embodiment, the autoantibodies are detected in serum.

The presence and/or level of a cancer-associated autoantibody in a sample from a subject can be determined in the presently disclosed methods. Representative autoantibody markers include but are not limited to autoantibodies against Complement Factor H (CFH), alpha-glucosidase (GANAB), STIP1 (Stress-induced-phosphoprotein 1), alpha-enolase, 14-3-3 (14-3-3 protein epsilon), and HSP 60 (60 kDa heat shock protein). Additional representative antibody markers include but are not limited to autoantibodies respectively against the entities listed in Tables 2-4, presented herein below. However, the presently disclosed subject matter is not limited to these autoantibodies. Any autoantibody that is associated with cancer or the progression of cancer can be included in a panel as provided herein, and is within the scope of the presently disclosed subject matter. Any suitable method can be utilized to identify additional cancer autoantibodies suitable for use in the presently disclosed methods, including but not limited to, the methods described in the illustrative Examples below and in Section II.C. below.

As is apparent from the foregoing embodiments, the presently disclosed methods and compositions are useful for screening subjects for cancer, for the early detection of cancer, and for managing the treatment of subjects with potential cancer or with known cancer. For example, the presently disclosed methods and compositions are useful for screening subjects prior to imaging or other known methods for detecting tumors, and are useful to define subjects at high risk or higher risk for cancer. For example those subjects with both a high-risk clinical profile and a test result from an autoantibody screen indicating a high or higher probability of lung cancer can be sent on to have a CT scan performed. Subjects whose test results suggest a low probability of cancer can be reevaluated using the autoantibody screen during their routine follow-up.

The presently disclosed methods and compositions provided herein can be employed where an indeterminate pulmonary nodule is detected on imaging studies, whether detected in a screening trial or performed for other indications. Those subjects with a test result from an autoantibody screen indicating a low risk of cancer can be followed with imaging studies at regular intervals. Subjects having both a high-risk clinical profile and an autoantibody screen result associated with a high risk of malignancy can be determined to require immediate intervention.

Any suitable method for determining the presence and/or level of a cancer-associated autoantibody as would be apparent to one skilled in the art upon a review of the present disclosure can be employed. For example, methods for detecting autoantibodies can include immunoblot with purified protein, an ELISA, and/or a protein assay.

In some embodiments, autoantibodies are detected using technologies well known to those of skill in the art such as gel electrophoresis and the use of a binding partner. For example, Complement Factor H (CFH), alpha-glucosidase (GANAB), STIP1 (Stress-induced-phosphoprotein 1), alpha-enolase, 14-3-3 (14-3-3 protein epsilon), and/or HSP 60 (60 kDa heat shock protein) polypeptides, or fragments thereof, can be used as binding partners. Additional representative binding partners include but are not limited to the entities listed in Tables 2-4, presented herein below.

Additionally, antibodies can be used as binding partners. An antibody of the presently disclosed subject matter can be any monoclonal or polyclonal antibody, so long as it suitably recognizes the desired target molecule. In some embodiments, antibodies are produced to an immunogen according to any conventional antibody or antiserum preparation process. In addition, a protein used herein as the immunogen is not limited to any particular type of immunogen. For example, fragments of the autoantibodies of the presently disclosed subject matter can be used as immunogens. The fragments can be obtained by any method including, but not limited to expressing a fragment of the gene encoding the protein, enzymatic processing of the protein, chemical synthesis, and the like.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc. Representative immunoassays are described in U.S. Pat. Nos. 5,599,677 and 5,672,480, each of which is herein incorporated by reference. Upon review of the present disclosure, including the Examples, those skilled in the art will be familiar with numerous specific immunoassay formats and variations thereof that will be useful for carrying out the methods of the presently disclosed subject matter.

In some embodiments, antibody binding to the autoantibodies is detected by detecting a label on the primary antibody. In some embodiments, the primary antibody is detected by detecting binding of a secondary antibody or reagent to the primary antibody: In some embodiments, the secondary antibody is labeled. Approaches for producing a detectable signal include the use of radioactive labels (e.g., ³⁵S, ¹²⁵I, ¹³¹I), fluorescent labels, enzyme labels (e.g., horseradish peroxidase, alkaline phosphatase), fluorescent labels (e.g., fluorescein) and so forth, in accordance with known techniques, as will be apparent to one skilled in the art upon review of the present disclosure. Many methods are known in the art for detecting binding in an immunoassay and are within the scope of the presently disclosed subject matter.

In some embodiments, an immunoassay comprises antibodies specific for the autoantibodies and approaches for producing a detectable signal. The antibodies can be immobilized on a support (such as a bead, plate or slide) in accordance with known techniques and contacted with a test sample in liquid phase. The support is then separated from the liquid phase and either the support phase or the liquid phase is examined for the detectable signal that is related to the presence of the autoantibody.

The presently disclosed subject matter includes kits for detecting the autoantibodies. In some embodiments, the kit comprises antibodies specific for a cancer-associated autoantibody, the reagents necessary for producing a detectable signal as described above and buffers. In some embodiments, the kit contains all of the components necessary to perform a detection assay, including all controls, directions for performing assays, and any necessary software for analysis and presentation of results.

Detection kits for carrying out the methods of the presently disclosed subject matter can be produced in a number of ways. In some embodiments, the detection kit comprises an antibody or antibody fragment that specifically binds to a cancer-associated autoantibody conjugated to a solid support and a second such antibody or antibody fragment conjugated to a detectable group. In some embodiments, the kit also includes ancillary reagents such as buffering agents and protein stabilizing agents, and can include (where necessary) other members of the detectable signal-producing system of which the detectable group is a part (e.g., enzyme substrates); agents for reducing background interference in a test; control reagents; apparatus for conducting a test, and the like, as will be apparent to those skilled in the art upon a review of the instant disclosure.

In some embodiments, the kit comprises antibodies or antibody fragments specific for one or more cancer-associated autoantibodies, and a specific binding partner for each of the antibodies that is conjugated to a detectable group. Ancillary agents as described above can likewise be included. The test kit can be packaged in any suitable manner, typically with all groups in a single container along with a sheet or printed instructions for carrying out the test.

In some embodiments, the detection assay for the autoantibodies is automated. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. In this manner, a clinician can access the test results using any suitable approach or device. Thus, in some embodiments, a clinician need not understand the raw data, as the data is presented directly to the clinician in its most useful form. The clinician is then able to immediately utilize the information to optimize care of the subject. The presently disclosed subject matter provides any method capable of receiving, processing, and transmitting the information to and from laboratories conducting the assays, information providers, medical personnel, and subjects.

II.B. Therapeutic Methods

Also provided in accordance with the presently disclosed subject matter are compositions and methods for treating cancer in a subject. The presently disclosed subject matter can comprise in some embodiments administering to a subject having a cancer an effective amount of an antibody (in some cases this can be a bispecific antibody) having the immunoreaction characteristics of a cancer-associated autoantibody, an antigen for a cancer-associated autoantibody, or both an antibody having the immunoreaction characteristics of a cancer-associated autoantibody and an antigen for a cancer-associated autoantibody.

In some embodiments, an antibody against Complement Factor H (CFH), an antibody against alpha-glucosidase (GANAB), an antibody against STIP1 (Stress-induced-phosphoprotein 1), an antibody against alpha-enolase, an antibody against 14-3-3 (14-3-3 protein epsilon), or an antibody against HSP 60 (60 kDa heat shock protein), or any combination of the aforementioned antibodies is/are administered. In some embodiments the administering can comprise administering a Complement Factor H (CFH) antigen, an alpha-glucosidase (GANAB) antigen, a STIP1 (Stress-induced-phosphoprotein 1) antigen, an alpha-enolase antigen, a 14-3-3 (14-3-3 protein epsilon) antigen, or a HSP 60 (60 kDa heat shock protein) antigen.

Additional representative antibodies that can be administered include but are not limited to antibodies respectively against the entities listed in Tables 2-4, presented herein below. Any combination of these antibodies can be administered, as can any combination of these antibodies with those listed in the immediately herein above. Additional representative antigens that can be administered include but are not limited to antigens prepared from the entities listed in Tables 2-4, presented herein below. Any combination of these antigens can be administered, as can any combination of these antigens with those listed in immediately herein above. Thus, in some embodiments any combination of the aforementioned antigens can be administered.

In some embodiments a bispecific antibody—such as but not limited to one part containing a complement inhibitory antibody (CFH, CD46, CD55, CD35, and CD59), combined with another tumor protein (e.g. EGFR) and/or an adjuvant is administered to the subject. Thus provided herein in some embodiments is therapy with bispecific antibodies using complement inhibitory proteins that can be combined with another antibody against a tumor protein.

In some embodiments a tissue to be treated is a cancerous tissue of a subject with a solid tumor, a metastasis, or other type of cancer. One exemplary type of cancer is lung cancer. Other example cancers include melanoma, adenocarcinoma, malignant glioma, prostatic carcinoma, kidney carcinoma, bladder carcinoma, pancreatic carcinoma, thyroid carcinoma, colon carcinoma, rectal carcinoma, brain carcinoma, liver carcinoma, breast carcinoma, ovary carcinoma, solid tumors, solid tumor metastases, angiofibromas, retrolental fibroplasia, hemangiomas, and Karposi's sarcoma. Additional exemplary cancers would be apparent to one of ordinary skill in the art upon a review of the instant disclosure.

In some embodiments, the presently disclosed subject matter pertains to the practice of the method in conjunction with other therapies such as conventional chemotherapy or surgery directed against solid tumors and for control of establishment of metastases. The administration of a therapeutic composition in accordance with the presently disclosed subject matter can be conducted before, during or after chemotherapy or surgery. For example, the presently disclosed subject matter can be practiced for chronic maintenance. As additional example, the presently disclosed subject matter can be practiced after a regimen of chemotherapy at times where the tumor tissue will be responding to toxic assault. As a further example, the presently disclosed subject matter can be practiced after surgery where solid tumors have been removed as a prophylaxis against metastases.

The dosage ranges for the administration of therapeutic agent can depend upon the form of the agent, and its potency, as described further herein, and are amounts large enough to produce the desired effect in which disease symptoms are ameliorated. The dosage should not be so large as to cause adverse side effects, such as hyperviscosity syndromes, pulmonary edema, congestive heart failure, and the like. Generally, the dosage will vary with the age, condition, sex and extent of the disease in the subject and can be determined by one of skill in the art. The dosage can also be adjusted by the individual physician in the event of any complication.

An effective amount can be an amount of an antibody having the immunoreaction characteristics of a cancer-associated autoantibody and/or an antigen for a cancer-associated autoantibody sufficient to produce a measurable effect in the tissue being treated. In some embodiments a desired effect can be the prevention of metastasis. As such, the treatment is not necessarily in a particular tissue, but rather can be a systemic treatment. Thus, in some embodiments, an effective amount is an amount of an antibody having the immunoreaction characteristics of a cancer-associated autoantibody and/or an antigen for a cancer-associated autoantibody sufficient to produce the maintenance of a desired state in the individual as a whole, such as but not limited to the absence of metastasis). Measurements can be made as described in the Example herein below or by other methods known to one skilled in the art.

The presently disclosed therapeutic compositions can be administered parenterally by injection or by gradual infusion over time. Although the tissue to be treated can typically be accessed in the body by systemic administration and therefore most often treated by intravenous administration of therapeutic compositions, other tissues and delivery approaches where there is a likelihood that the tissue targeted contains the target molecule. Thus, therapeutic compositions can be inhaled or otherwise provided to the lungs, and/or can be administered intravenously, intraperitoneally, intramuscularly, subcutaneously, intra-cavity, transdermally, and can be delivered by peristaltic approaches.

The therapeutic compositions of the presently disclosed subject matter are conventionally administered intravenously, as by injection of a unit dose, for example. The term “unit dose” when used in reference to a therapeutic compositions of the presently disclosed subject can refer to physically discrete units suitable as unitary dosage for the subject, each unit containing a predetermined quantity of active material calculated to produce the desired therapeutic effect in association with the required diluent; i.e., carrier or vehicle.

The compositions are administered in a manner compatible with the dosage formulation, and in an effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. Suitable regimes for administration are also variable, but are typified by an initial administration followed by repeated doses at one or more hour intervals by a subsequent injection or other administration.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. In some embodiments, the pharmaceutically acceptable carrier is pharmaceutically acceptable in humans.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use can also be prepared. The preparation can also be emulsified.

The active ingredient can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

II.B.1. Antigenic Polypeptides

In some embodiments, the presently disclosed subject provides therapeutic compositions comprising an antigenic polypeptide for a cancer-associated autoantibody. Representative antigenic polypeptides include Complement Factor H (CFH), alpha-glucosidase (GANAB), STIP1 (Stress-induced-phosphoprotein 1), alpha-enolase, 14-3-3 (14-3-3 protein epsilon), HSP 60 (60 kDa heat shock protein), or fragments or analogs thereof. Additional representative antigenic polypeptides include but are not limited to the entities listed in Tables 2-4, presented herein below.

In one embodiment, a polypeptide of the presently disclosed subject matter comprises no more than about 100 amino acid residues, preferably no more than about 60 residues, more preferably no more than about 30 residues. Peptides can be linear or cyclic. Thus, it should be understood that a subject polypeptide need not be identical to a native amino acid residue sequence of an antigenic polypeptide, so long as it includes required sequences to generate an immune response.

A subject polypeptide includes any analog, fragment or chemical derivative of an antigenic polypeptide for a cancer-associated autoantibody. Such a polypeptide can be subject to various changes, substitutions, insertions, and deletions where such changes provide for certain advantages in its use. In this regard, an antigenic polypeptide for a cancer-associated autoantibody corresponds to, rather than is identical to, the sequence of the native antigen where one or more changes are made and it retains the ability to function as an antigenic polypeptide for a cancer-associated autoantibody in one or more of the assays as defined herein. Thus, a polypeptide can be in any of a variety of forms of peptide derivatives, that include amides, conjugates with proteins, cyclized peptides, polymerized peptides, analogs, fragments, chemically modified peptides, and the like derivatives.

The term “analog” includes any polypeptide having an amino acid residue sequence substantially identical to a sequence of an antigenic polypeptide for a cancer-associated autoantibody in which one or more residues have been conservatively substituted with a functionally similar residue and which displays antigenic activity as described herein. Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

The phrase “conservative substitution” also includes the use of a chemically derivatized residue in place of a non-derivatized residue provided that such polypeptide displays the requisite inhibition activity.

“Chemical derivative” refers to a subject polypeptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine. Also included as chemical derivatives are those peptides that contain one or more naturally occurring amino acid derivatives of the twenty standard amino acids. For examples: 4-hydroxyproline can be substituted for proline; 5-hydroxylysine can be substituted for lysine; 3-methylhistidine can be substituted for histidine; homoserine can be substituted for serine; and ornithine can be substituted for lysine. Polypeptides of the presently disclosed subject matter also include any polypeptide having one or more additions and/or deletions or residues relative to the sequence of a polypeptide whose sequence is shown herein, so long as the requisite activity is maintained. The term “fragment” refers to any subject polypeptide having an amino acid residue sequence shorter than that of a polypeptide disclosed herein.

When a polypeptide of the presently disclosed subject matter has a sequence that is not identical to the sequence of an antigenic polypeptide for a cancer-associated autoantibody, it is typically because one or more conservative or non-conservative substitutions have been made, usually when no more than about 30 number percent, and preferably when no more than 10 number percent of the amino acid residues are substituted. Additional residues can also be added at either terminus of a polypeptide for the purpose of providing a “linker” by which the polypeptides of the presently disclosed subject matter can be conveniently affixed to a label or solid matrix, or carrier.

Amino acid residue linkers are usually at least one residue and can be 40 or more residues, more often 1 to 10 residues, but do not form antigenic epitopes. Typical amino acid residues used for linking are tyrosine, cysteine, lysine, glutamic and aspartic acid, or the like. In addition, a subject polypeptide can differ, unless otherwise specified, from an antigenic polypeptide for a cancer-associated autoantibody by the sequence being modified by terminal-NH2 acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong the half life of the polypeptides in solutions, particularly biological fluids where proteases can be present. In this regard, polypeptide cyclization is also a useful terminal modification, and can be beneficial also because of the stable structures formed by cyclization.

Any peptide of the presently disclosed subject matter can be used in the form of a pharmaceutically acceptable salt. Suitable acids which are capable of reacting with a peptide of the presently disclosed subject matter include inorganic acids such as trifluoroacetic acid (TFA), hydrochloric acid (HCl), hydrobromic acid, perchioric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Suitable bases capable of forming salts with the peptides of the presently disclosed subject matter include inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine and the like), and optionally substituted ethanolamines (e.g. ethanolamine, diethanolamine and the like).

A peptide of the presently disclosed subject matter, also referred to herein as a subject polypeptide, can be synthesized by any of the techniques that are known to those skilled in the polypeptide art, including recombinant DNA techniques. Synthetic chemistry techniques, such as a solid-phase Merrifield-type synthesis, can be employed for reasons of purity, antigenic specificity, freedom from undesired side products, ease of production and the like. A summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv Enzymol, 32:221-96, 1969; Fields et al., Int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemistry”, Plenum Press, New York, 1973, which is incorporated herein by reference.

In general, the solid-phase synthesis methods can comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as exemplary, the protected or derivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to afford the final linear polypeptide.

The resultant linear polypeptides prepared for example as described above can be reacted to form their corresponding cyclic peptides. An exemplary method for cyclizing peptides is described by Zimmer et al., Peptides 1992, pp. 393-394, ESCOM Science Publishers, B. V., 1993. Typically, tertbutoxycarbonyl protected peptide methyl ester is dissolved in methanol and sodium hydroxide solution are added and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

II.B.2. Antibodies

The presently disclosed subject matter also provides in some embodiments a cancer-associated autoantibody, as well as methods for isolating the autoantibody and/or for producing an antibody with the same immunoreaction characteristics (for example, antigen recognition properties) as the autoantibody. An antibody with such properties can be referred to as an “autoantibody mimic”.

Thus, the phrase, “an antibody having the immunoreaction characteristics of a cancer-associated autoantibody” can include but is not limited to a cancer-associated autoantibody as disclosed herein itself, or an autoantibody mimic. Further, an antibody having the immunoreaction characteristics of a cancer-associated autoantibody can be a bispecific antibody as described herein above.

Representative methods for producing autoantibody mimics and for isolating autoantibodies are provided herein, including in the Examples. Representative antibodies include but are not limited to antibodies respectively against Complement Factor H (CFH), alpha-glucosidase (GANAB), STIP1 (Stress-induced-phosphoprotein 1), alpha-enolase, 14-3-3 (14-3-3 protein epsilon), and HSP 60 (60 kDa heat shock protein). Additional representative antibodies include but are not limited to antibodies respectively against the entities listed in Tables 2-4, presented herein below.

The term “antibody or antibody molecule” in the various grammatical forms is used herein as a collective noun that refers to a population of immunoglobulin molecules and/or immunologically active portions of immunoglobulin molecules, i.e., molecules that contain an antibody combining site or paratope. An “antibody combining site” is that structural portion of an antibody molecule comprised of heavy and light chain variable and hypervariable regions that specifically binds antigen. Also included are heavy chain only antibodies and antibody fragments obtained from camelids (e.g. llamas). A general technique for the production of such antibodies and antibody fragments is provided in Frenken, Leon G. J., et al., Journal of Biotechnology 78 (2000) 11-21, herein incorporated by reference in its entirety.

Exemplary antibodies for use in accordance with the presently disclosed subject matter are intact immunoglobulin molecules, substantially intact immunoglobulin molecules, single chain immunoglobulins or antibodies, those portions of an immunoglobulin molecule that contain the paratope, including those portions known in the art as Fab, Fab′, F(ab′)₂ and F(v), and also referred to as antibody fragments.

The phrase “monoclonal antibody” in its various grammatical forms refers to a population of antibody molecules that contain only one species of antibody combining site capable of immunoreacting with a particular epitope. A monoclonal antibody thus typically displays a single binding affinity for any epitope with which it immunoreacts. A monoclonal antibody can therefore contain an antibody molecule having a plurality of antibody combining sites, each immunospecific for a different epitope, e.g., a bispecific monoclonal antibody.

A monoclonal antibody is typically composed of antibodies produced by clones of a single cell called a hybridoma that secretes (produces) only one kind of antibody molecule. The hybridoma cell is formed by fusing an antibody-producing cell and a myeloma or other self-perpetuating cell line. The preparation of such antibodies was first described by Kohler and Milstein, Nature 256:495-497 (1975), which description is incorporated by reference. Additional methods are described by Zola, Monoclonal Antibodies: A Manual of Techniques, CRC Press, Inc. (1987). The hybridoma supernates so prepared can be screened for the presence of antibody molecules (i.e. autoantibody mimics) that immunoreact with an antigen for a cancer-associated autoantibody.

Briefly, to form the hybridoma from which the monoclonal antibody composition is produced, a myeloma or other self-perpetuating cell line is fused with lymphocytes obtained from the spleen of a mammal hyperimmunized with a source of an antigen, as described by Cheresh et al., J. Biol Chem, 262:17703-17711 (1987).

It is suggested that the myeloma cell line used to prepare a hybridoma be from the same species as the lymphocytes. Typically, a mouse of the strain 129 GIX+ is a typical mammal. Representative mouse myelomas for use in the presently disclosed subject matter include the hypoxanthine-aminopterin-thymidine-sensitive (HAT) cell lines P3X63-Ag8.653, and Sp2/0-Ag14 that are available from the ATCC, Manassas, Va., under the designations CRL 1580 and CRL 1581, respectively.

Splenocytes are typically fused with myeloma cells using polyethylene glycol (PEG) 1500. Fused hybrids are selected by their sensitivity to HAT. Hybridomas producing a monoclonal antibody of the presently disclosed subject matter are identified using the enzyme linked immunosorbent assay (ELISA) described in the Examples.

A monoclonal antibody of the presently disclosed subject matter can also be produced by initiating a monoclonal hybridoma culture comprising a nutrient medium containing a hybridoma that secretes antibody molecules of the appropriate specificity. The culture is maintained under conditions and for a time period sufficient for the hybridoma to secrete the antibody molecules into the medium. The antibody-containing medium is then collected. The antibody molecules can then be further isolated by well known techniques. Media useful for the preparation of these compositions are both well known in the art and commercially available and include synthetic culture media, inbred mice and the like. An exemplary synthetic medium is

Dulbecco's minimal essential medium (DMEM—Dulbecco et al., Virol 8:396 (1959)) supplemented with 4.5 gm/1 glucose, 20 mM glutamine, and 20% fetal calf serum. An exemplary inbred mouse strain is the Balb/C.

Other methods of producing a monoclonal antibody, a hybridoma cell, or a hybridoma cell culture are also well known. See, for example, the method of isolating monoclonal antibodies from an immunological repertoire as described by Sastry, et al., Proc Natl Acad Sci USA 86:5728-5732 (1989); and Huse et al., Science 246:1275-1281 (1989).

Also provided by the presently disclosed subject matter is the hybridoma cell, and cultures containing a hybridoma cell that produce a monoclonal antibody of the presently disclosed subject matter. The presently disclosed subject matter thus provides in some embodiments, a monoclonal antibody that has the immunoreaction characteristics (i.e. an autoantibody mimic) of a cancer-associated autoantibody as described in the Examples.

It is also possible to determine, without undue experimentation, if a monoclonal antibody has the same (i.e., equivalent) specificity (immunoreaction characteristics) as a monoclonal antibody of the presently disclosed subject matter by ascertaining whether the former prevents the latter from binding to a preselected target molecule. If the monoclonal antibody being tested competes with the monoclonal antibody of the presently disclosed subject matter, as shown by a decrease in binding by the monoclonal antibody of the presently disclosed subject matter in standard competition assays for binding to the target molecule when present in the solid phase, then it is likely that the two monoclonal antibodies bind to the same, or a closely related, epitope.

Still another way to determine whether a monoclonal antibody has the specificity of a monoclonal antibody of the presently disclosed subject matter is to pre-incubate the monoclonal antibody of the presently disclosed subject matter with the target molecule with which it is normally reactive, and then add the monoclonal antibody being tested to determine if the monoclonal antibody being tested is inhibited in its ability to bind the target molecule. If the monoclonal antibody being tested is inhibited then, in all likelihood, it has the same, or functionally equivalent, epitopic specificity as the monoclonal antibody of the presently disclosed subject matter.

An additional way to determine whether a monoclonal antibody has the specificity of a monoclonal antibody of the presently disclosed subject matter is to determine the amino acid residue sequence of the CDR regions of the antibodies in question. Antibody molecules having identical, or functionally equivalent, amino acid residue sequences in their CDR regions have the same binding specificity. Methods for sequencing polypeptides are known in the art.

The immunospecificity of an antibody, its target molecule binding capacity, and the attendant affinity the antibody exhibits for the epitope, are defined by the epitope with which the antibody immunoreacts. The epitope specificity is defined at least in part by the amino acid residue sequence of the variable region of the heavy chain of the immunoglobulin that comprises the antibody, and in part by the light chain variable region amino acid residue sequence. Use of the terms “having the binding specificity of” or “having the binding preference of” indicates that equivalent monoclonal antibodies exhibit the same or similar immunoreaction (which can include binding) characteristics and compete for binding to a preselected target molecule.

Humanized monoclonal antibodies offer particular advantages over murine monoclonal antibodies, particularly insofar as they can be used therapeutically in humans. Specifically, human antibodies are not cleared from the circulation as rapidly as “foreign” antigens, and do not activate the immune system in the same manner as foreign antigens and foreign antibodies. Methods of preparing “humanized” antibodies are generally well known in the art, and can readily be applied to the antibodies of the presently disclosed subject matter. Thus, the presently disclosed subject matter provides in some embodiments that a monoclonal antibody of the presently disclosed subject matter that is humanized by grafting to introduce components of the human immune system without substantially interfering with the ability of the antibody to bind antigen. Humanized antibodies can also be produced using animals engineering to produce humanized antibodies, such as those available from Medarex of Annandale, N.J., United States of America (mice) and Abgenix, Inc., of Fremont, Calif., United States of America (mice).

The use of a molecular cloning approach to generate antibodies, particularly monoclonal antibodies, and more particularly single chain monoclonal antibodies, is also provided herein. The production of single chain antibodies has been described in the art, see e.g., U.S. Pat. No. 5,260,203, the contents of which are herein incorporated by reference. For this, combinatorial immunoglobulin phagemid or phage-displayed libraries are prepared from RNA isolated from the spleen of the immunized animal, and phagemids expressing appropriate antibodies are selected by panning on endothelial tissue. This approach can also be used to prepare humanized antibodies. The advantages of this approach over conventional hybridoma techniques are that approximately 10⁴ times as many antibodies can be produced and screened in a single round, and that new specificities are generated by H and L chain combination in a single chain, which further increases the chance of finding appropriate antibodies. Thus, an antibody of the presently disclosed subject matter, or a “derivative” of an antibody of the presently disclosed subject matter pertains to a single polypeptide chain binding molecule which has binding specificity and affinity substantially similar to the binding specificity and affinity of the light and heavy chain aggregate variable region of an antibody described herein.

“Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. For a review of scFv see Pluckthun, in The Pharmacology of Monoclonal Antibodies, vol. 113, Rosenburg and Moore eds., Springer-Verlag, N.Y., pp. 269-315 (1994).

In some embodiments, provided herein is the preparation of antibody phage display libraries as a tool to discover novel autoantibody antigens. Using mRNA from peripheral blood lymphocytes or lymph nodes from pools of lung cancer patients having tumors of various stages and histologies, antibody phage display libraries are prepared that are then screened against tumor (for example lung tumor proteins) with the aim of identifying phage antibodies that bind to tumor proteins. The phage-expressed antibodies are then used to identify the antigen proteins using affinity capture or immunoblot strategies. The preparation and utilization of these cancer antibody phage libraries provides a replenishable resource. Identified antigen polypeptides are then validated by determining the prevalence of autoantibodies against these antigens in the serum of a population of cancer patients and non-cancer controls. This validation step will ensure that all antigens to be used in the subsequent serum autoantibody test are indeed cancer (such as but not limited to lung cancer) autoantibodies as opposed to proteins bound fortuitously by phage antibodies in the libraries. The methods can be carried out by incubating patients' serum on antigen microarrays containing a panel of validated tumor autoantigens and then determining the pattern of immunoreactivity using one of various methods of detecting bound human immunoglobulins. The pattern of immunoreactivity can serve as an autoantibody signature that can indicate the presence of cancer, such as but not limited to lung cancer.

### III. EXAMPLES

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

### Overview of Examples

The following Examples pertain to the discovery of functional autoantibodies in early stage lung cancer patients.

Lung cancer is the most common cancer, and the overall 5-year survival is only 15% as the majority of patients present with advanced stage disease. As part of an early detection strategy these Examples pertain to the discovery of molecular markers that could detect early stage disease and to the pursuit of novel therapeutic targets. A search was conducted for autoantibodies present in stage I lung cancer patients that are not present in patients with advanced stage disease. In Examples 1-4 it is shown that 14 of 28 stage I non-recurrent NSCLC patients (50%) were positive for an autoantibody to Complement Factor H (CFH) while only 3 of 28 late stage NSCLC patients had the autoantibody (p=0.003). CFH is a complement protective protein that inactivates factor C3b whose deposition leads to formation of a cytolytic attack complex. Addition of IgG from patients positive for anti-CFH antibodies to A549 lung adenocarcinoma cells led to increased complement activation on the cell surface compared to IgG from patients lacking anti-CFH antibodies (p=0.01). These findings suggest for the first time that the CFH autoantibody is a molecular marker of early stage lung cancer, and could play a role in suppressing tumor growth by increasing complement activation on tumor cells.

Example 5 pertains to the discovery of alpha-glucosidase (GANAB) autoantibodies in cancer patients and provides another example of a molecular marker for cancer.

Examples 6 and 7 pertain to the discovery of additional autoantibodies and provide additional examples of molecular markers for cancer.

### Example 1

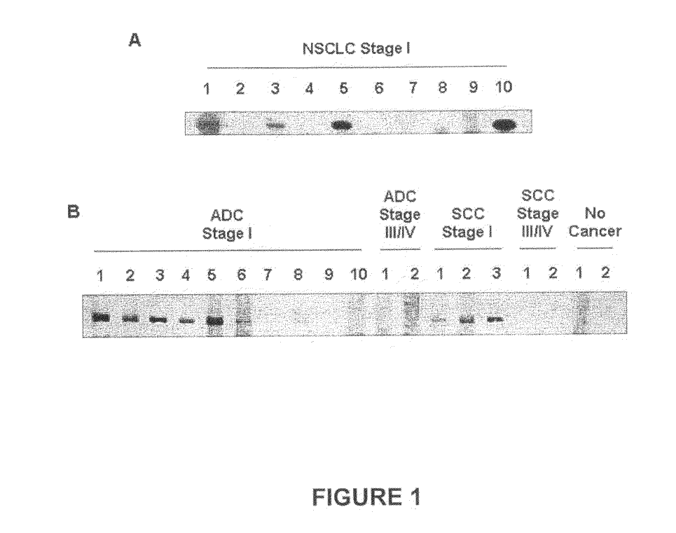

Although monoclonal antibodies are used as therapeutic agents in many malignancies, the host immune response to tumors is thought to be primarily modulated by cellular immunity. O. J. Finn, J Immunol 178, 2615 (Mar. 1, 2007). Humoral immunity in NSCLC and in most tumors is not well characterized and autoantibodies are not typically associated with a distinct clinical phenotype. It was hypothesized that early stage lung cancer patients might have autoantibodies to tumor cell proteins that advanced stage patients might not, and that these autoantibodies might have a functional role in limiting metastasis. As the first step in addressing this question, an immunoblot containing the pooled sera of 5 patients with advanced stage NSCLC was prepared. The blot was probed with individual serum samples from a group of 10 patients with early stage NSCLC who had not relapsed in at least 2 years. The immunoblot was probed with a secondary anti-human IgG horseradish peroxidase (HRP) conjugate in order to detect antibody-antigen complexes. Although several different immunoreactive bands were detected, a 150 kDa band was seen in. 4 of the 10 patients with early stage disease (FIG. 1A).

The immunoreactive 150 kDa band on the blot was identified by alignment of an immunoblot displaying the band with an adjacent gel lane that had been stained with Coomassie blue, followed by excision of the band, and subjection of the protein to in-gel tryptic digestion, MALDI-TOF peptide fingerprint analysis and MS/MS sequencing. The sequence analysis identified the band to be a mixture of ceruloplasmin and complement factors 3, 4A, and H. Immunoblot analysis was used against purified ceruloplasmin, and complement factors 3 and H to identify CFH as the immunoreactive protein. CFH is a human plasma protein that inhibits the formation and activity of the C3 convertase in the complement cascade. S. Rodriguez de Cordoba, J. Esparza-Gordillo, E. Goicoechea de Jorge, M. Lopez-Trascasa, P. Sanchez-Corral, Mol Immunol 41, 355 (June 2004). A prior study had shown CFH to be secreted and bound to the membrane of some NSCLC cell lines, functioning as one of the protective proteins defending tumor cells against complement mediated cytotoxicity. D. Ajona et al., Cancer Res 64, 6310 (Sep. 1, 2004); D. Ajona, Y. F. Hsu, L. Corrales, L. M. Montuenga, R. Pio, J Immunol 178, 5991 (May 1, 2007). This is the first report of an autoantibody to CFH in early stage NSCLC.

### Example 2

In order to determine if the CFH autoantibody could be used to differentiate early from late stage patients, purified CFH was run on a gel, blotted to a membrane, and the membrane was probed with sera from 28 stage I patients, 28 stage III/IV patients, and sera from 12 control patients without cancer. A representative sample of the results is shown (FIG. 1B). Overall, 14 of 28 non-recurrent stage I NSCLC patients (50%) were positive for the CFH autoantibody, including 10 of 17 adenocarcinoma (ADC) and 4 of 11 squamous cell carcinoma (SCC) patients, while 3 of 28 late stage NSCLC patients had the autoantibody (10.7%) including 2 of 16 with ADC and 1 of 12 with SCC. The difference in the incidence of CFH autoantibody in early vs. late stage NSCLC was statistically significant (p=0.003). None of the 12 individuals without cancer had the CFH autoantibody.

### Example 3

CFH is a complement inhibitory protein produced and secreted by the liver, monocytes and macrophages. It has also been found to be associated with cancer cells, including those of lung, colon, ovarian, bladder and glial origin. D. Ajona et al., Cancer Res 64, 6310 (Sep. 1, 2004); D. Ajona, Y. F. Hsu, L. Corrales, L. M. Montuenga, R. Pio, J Immunol 178, 5991 (May 1, 2007); E. Wilczek et al., Int J Cancer 122, 2030 (May 1, 2008); S. Junnikkala et al., Br J Cancer 87, 1119 (Nov 4, 2002); Z. Z. Cheng et al., Clin Chem 51, 856 (May, 2005); P. Gasque, A. Thomas, M. Fontaine, B. P. Morgan, J Neuroimmunol 66, 29 (May, 1996); S. Junnikkala et al., J Immunol 164, 6075 (Jun. 1, 2000). There is evidence that CFH and other complement regulatory proteins, including CD35 (CR1), CD46 (MCP), CD55 (DAF), and CD59 (protectin), protect tumor cells from attack by the complement system. K. Jurianz et al., Mol Immunol 36, 929 (Sep-Oct, 1999). CFH inhibits the alternative complement pathway by binding to a critical complement component, C3b, in both the fluid phase and at the membrane. S. Rodriguez de Cordoba, J. Esparza-Gordillo, E. Goicoechea de Jorge, M. Lopez-Trascasa, P. Sanchez-Corral, Mol Immunol 41, 355 (June 2004). It acts as a cofactor for the factor I-mediated proteolysis and inactivation of C3b, and accelerates the decay of the C3bBb convertase that generates

C3b at the membrane. S. Rodriguez de Cordoba, J. Esparza-Gordillo, E. Goicoechea de Jorge, M. Lopez-Trascasa, P. Sanchez-Corral, Mol Immunol 41, 355 (Jun, 2004). Deposition of C3b initiates the formation of the cell-lytic membrane attack complex. K. M. Murphy, P. Travers, M. Walport, Janeway's Immunobiology (Garland Science, London, England, ed. 7th, 2007).

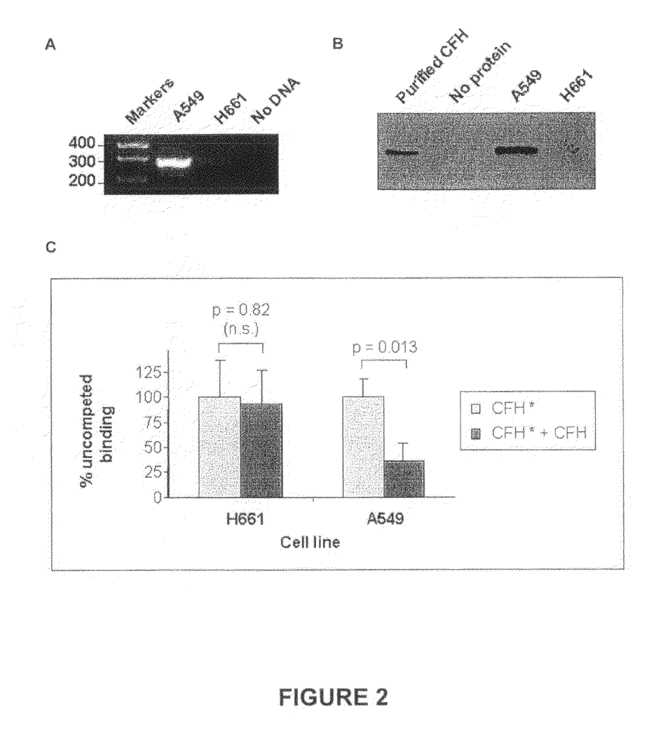

To examine whether the CFH autoantibody found in NSCLC patients is able to increase deposition of C3b on cell membranes, a C3 deposition assay was used with two lung cancer cell lines. D. Ajona et al., Cancer Res 64, 6310 (Sep. 1, 2004). This assay uses a radiolabeled monoclonal antibody that recognizes C3, C3b, and iC3b, a cleavage product of C3b. As a prerequisite to this experiment, previous observations were confirmed (D. Ajona et al., Cancer Res 64, 6310 (Sep. 1, 2004)) that the A549 cell line expresses (FIG. 2A), secretes (FIG. 2B), and binds (FIG. 2C) CFH while the H661 cell line does not and thus can be used as a negative control.

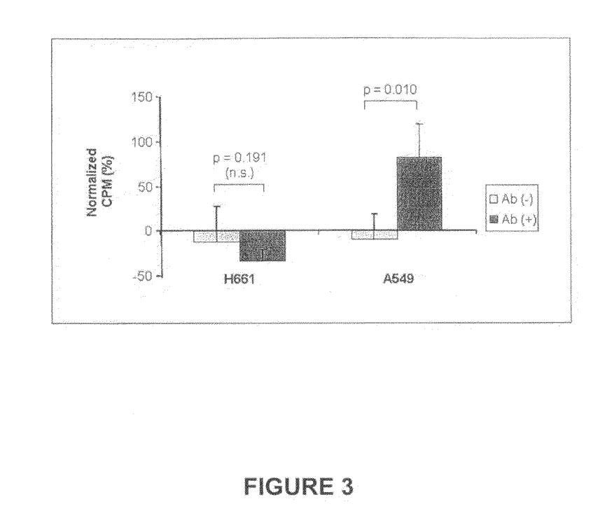

Three (3) patients with early stage disease who had the CFH autoantibody (2 patients with ADC and one patient with SCC) and 3 with advanced staged disease without the autoantibody (2 patients with ADC and one patient with SCC) were then selected. The IgG fraction was extracted from each of their sera, and independently spiked into a dilution of normal, control serum (used as a source of complement); half of each sample was then added to A549 cells and half to H661 cells. IgG from patients with the CFH autoantibodies showed significantly increased deposition of C3 on the A549 cell surface as compared to IgG from patients without the CFH autoantibody (p=0.01). In the H661 negative control cell line, C3 deposition in the presence of the CFH autoantibody was indistinguishable from that in its absence (p=0.19). (FIG. 3)

### Example 4

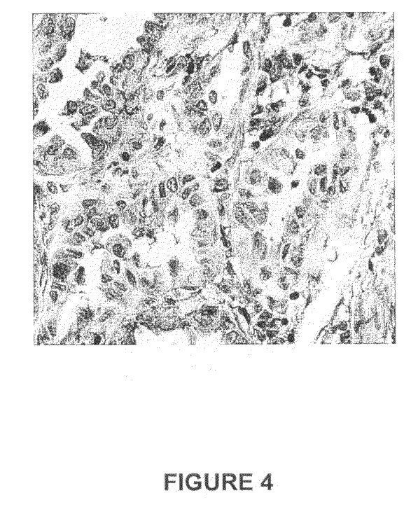

The primary function of CFH is to inhibit complement-mediated lysis by accelerating the removal of C3b and the inactivation of the C3 convertase C3bBb. S. Rodriguez de Cordoba, J. Esparza-Gordillo, E. Goicoechea de Jorge, M. Lopez-Trascasa, P. Sanchez-Corral, Mol Immunol 41, 355 (June 2004). It has been shown that neutralizing antibodies to complement inhibitory proteins increase C3b deposition and, in combination with antibodies against other complement protective proteins, increase cytotoxicity in cell lines that express and bind the protein. K. Jurianz et al., Mol Immunol 36, 929 (Sep-Oct, 1999). The reason why some patients develop anti-CFH antibodies and some do not is an open question. Previous studies have described CFH autoantibodies in patients with hemolytic uremia syndrome (HUS). M. A. Dragon-Durey et al., J Am Soc Nephrol 16, 555 (February 2005). None of the patients in this study had any renal disease, suggesting that the CFH autoantibody in lung cancer patients recognizes a different epitope from that recognized in HUS. Immunohistochemistry of 8 tumors, half from patients with and half from patients without the CFH autoantibody, showed diffuse tumor cell staining in all samples. A representative section of a lung adenocarcinoma immunostained for CFH is shown in FIG. 4. The fact that all the tumors examined express CFH but not all patients develop antibodies implies that mere expression of CFH is not enough to generate an antibody response in patients, and suggests that how the antigen is presented to the immune system is important.

### Discussion of Examples 1-4

Early detection of cancer, such as lung cancer, remains a difficult problem. Novel molecular markers that could not only detect and phenotype disease but that could also assist in determining appropriate targeted therapy would be of enormous clinical benefit. We chose to look at autoantibodies in NSCLC patients for two reasons. First, monoclonal antibodies are used to treat tumors, and we hypothesized that some patients might use their own antibodies to modulate tumor behavior. Second, autoantibodies could theoretically identify specific, abnormal targets and thus could be used diagnostically. This is the first study to find autoantibodies that point to a possible mechanism of tumor inhibition.

In Examples 1-4, half of patients with early stage lung cancer had CFH autoantibodies, a finding that suggests CFH autoantibodies can be used as a marker for early stage disease. The data also show that the CFH autoantibody caused increased deposition of C3-related protein on tumor cells, but prior reports have suggested that inactivation of a single complement inhibitory protein is not sufficient to cause cytotoxicity. D. Ajona, Y. F. Hsu, L. Corrales, L. M. Montuenga, R. Pio, J Immunol 178, 5991 (May 1, 2007); S. Junnikkala et al., J Immunol 164, 6075 (Jun. 1, 2000); S.

Varsano, L. Rashkovsky, H. Shapiro, D. Ophir, T. Mark-Bentankur, Clin Exp Immunol 113, 173 (August 1998). The CFH autoantibody is thus believed to play a role in a host defense mechanism resulting in limiting tumor growth and suppression of metastasis.

### Materials and Methods Employed in Examples 1-4

Patients. For the initial autoantibody screen, the serum samples of 5 stage III or IV NSCLC patients were pooled and this pool was immunoblotted as described below. Sera from another 10 early stage NSCLC patients, including 4 male and 6 female, with a mean age of 66.6 years (range 57-72 years) were individually used to probe the blot. For the CFH immunoblot survey of lung cancer patients, the individual serum samples of 28 patients were used, 14 males and 14 females with a mean age of 65.5 years (range 51-78 years) with stage I NSCLC, and 28 patients, 16 males and 12 females with a mean age of 64 years (range 41-83 years) with stage III or IV NSCLC. Another 12 control patients with no cancer were also enrolled. All cancer patients selected for entry into the study met the following criteria: 1) histologically confirmed NSCLC; 2) no prior chemotherapy or radiation for lung cancer; and 3) for early stage patients, no evidence of recurrence for at least 2 years following diagnosis. All sera were obtained prior to therapy at Duke University Medical Center, Durham, North Carolina, United States of America. The Institutional Review Board obtained informed consent from patients before the collection of biospecimens for medical research. Sera were frozen in liquid nitrogen and stored at −80° C.

Immunoblot analysis. For analysis of human serum from late stage NSCLC patients, a 15 μl aliquot of the serum pool was first processed with an Albumin and IgG Depletion Kit (GE Healthcare, Piscataway, N.J., United States of America) according to the manufacturer's instructions. The proteins (400 μg) in the depleted serum were separated under reducing conditions by SDS-PAGE in a 10% polyacrylamide gel containing a single preparative well, and then transferred to a polyvinylidene difluoride (PVDF) membrane. The PVDF membrane was placed in a Surf-Blot apparatus (Idea Scientific Company, Minneapolis, Minn., United States of America) that creates channels on the blot for multiple samples. For analysis of purified protein, 0.5 μg aliquots of purified human CFH (Complement Technology Inc., Tyler, Tex., United States of America) were run in multiple wells of a gel and transferred to PVDF. Patient sera were diluted 1:1000 dilution in 50 mM Tris-HCl, 137 mM NaCl, pH 7.6 and 5% (w/v) nonfat dry milk (MTBS) for probing blots. Goat anti-human IgGγ chain-HRP (Santa Cruz Biotechnology, Inc., Santa Cruz, Calif., United States of America) was used as the second antibody and was diluted 1:40,000 in MTBS. Bound antibody was detected using SuperSignal West Femto chemiluminescent detection system (Pierce, Rockford, Ill., United States of America).

Identification of the immunoreactive protein. Two aliquots of pooled late stage sera, depleted of albumin and IgG, were run in adjacent lanes of an SDS-PAGE gel under nonreducing conditions. Nonreducing conditions were necessary since the abundant serum protein alpha-2 macroglobulin runs at approximately the same molecular mass as the band of interest under reducing conditions, but at ˜320 kDa under nonreducing conditions. R. Sayegh, J. T. Awwad, C. Maxwell, B. Lessey, K. Isaacson, J Clin Endocrinol Metab 80, 1021 (March 1995). The proteins in one of the lanes were transferred to PVDF for immunoblot analysis, and the proteins in the other lane were stained with Coomassie blue. Immunoblot analysis was performed using serum #5 from FIG. 1A at 1:1000 dilution and goat anti-human γ-chain IgG-HRP at 1:40,000 dilution. Detection of immune complexes was with Luminol Western Blot HRP Substrate (Boston Bioproducts, Worcester, Mass., United States of America). The immunoblot was used to identify a band on the Coomassie stained gel running at the same molecular mass. The proteins in the band were then subjected to in-gel tryptic digestion and identified by MALDI-TOF peptide fingerprint analysis and MS/MS sequencing. Ceruloplasmin and complement factors 3, 4A, and H were identified from the excised band. Serum #5 was used to probe immunoblots containing purified ceruloplasmin and complement factors 3 and H, and CFH was identified as the immunoreactive protein.

Cancer cell lines. A549 (lung adenocarcinoma) and H661 (lung large cell carcinoma) cell lines were obtained from American Type Culture Collection. A549 was maintained in F-12K medium supplemented with 10% FBS, and H661 in RPMI 1640 with Glutamax medium supplemented with 10% FBS (Invitrogen, Carlsbad, Calif., United States of America). Both cell lines were maintained at 37° C. in 5% CO₂/95% air.

CFH mRNA expression by cell lines. Total RNA was isolated from A549 and H661 cells using a High Pure RNA Isolation Kit (Roche Applied Science, Indianapolis, Ind., United States of America) and cDNA was synthesized from 1 μg RNA using Transcriptor reverse transcriptase (Roche Applied Science). PCR was carried out with 0.5 μl of the cDNA (out of 20 μl cDNA reaction volume) or 0.5 μl water, CFH-specific primers and the Apex Taq MasterMix (Genesee Scientific, San Diego, Calif., United States of America) in a total volume of 50 μl. The sequence of the forward primer was 5′-GGAACCACCTCAATGCAAAG-3′ (SEQ ID NO:1) and the sequence of the reverse primer was 5′-AAGCTTCTGTTTGGCTGTCC-3′ (SEQ ID NO:2). The cycling conditions were as follows: initial denaturation for 2 minutes (min) at 94° C., followed by 30 cycles at 94° C. for 15 seconds (s), 52° C. for 30 s, and 72° C. for 45 s. Aliquots of the reactions were run on a 1.7% (w/v) TAE-agarose gel and visualized with GelStar (Lonza, Rockland, Me., United States of America). The expected amplicon size was 277 by and the product was verified as CFH DNA by sequence analysis.

CFH in conditioned media. A549 and H661 cells were grown to 80% confluence, the media were aspirated and the cells were washed 3 times with PBS and 2 times with serum-free media. Serum-free medium (10 ml) was added to each 75 cm² flask and the cells were incubated for 26.5 h at 37° C./5% CO₂. The media were harvested, residual cells were removed by centrifugation, and the media were concentrated 10-fold using a 10,000 MWCO centrifugal concentrator, and a further 5-fold using a 30,000 MWCO centrifugal concentrator (Sartorius, Goettingen, Germany). The total protein concentration was determined and 12 μg protein from each cell line was separated by SDS-PAGE alongside 100 ng purified human CFH. The proteins were blotted to PVDF and probed with goat anti-human CFH (Santa Cruz Biotechnology, Inc., Santa Cruz, Calif., United States of America), at 0.2 μg/ml in MTBS. The secondary antibody was donkey anti-goat IgG-HRP (Santa Cruz) at 1:20,000 dilution. Detection was with SuperSignal West Femto substrate (Pierce).

CFH Cell Binding Assay. A549 and H661 cells were grown to 80% confluence in 75-cm² flasks and detached with 0.25% Trypsin-EDTA (Sigma-Aldrich, St. Louis, Mo., United States of America). Cells were collected and resuspended at a concentration of 2×10⁶/ml in 50 μl binding buffer (1% BSA, 0.1% sodium azide in PBS). Purified CFH (Complement Technology, Inc.) was labeled with ¹²⁵I using lodobeads (Pierce, Rockford, Ill., United States of America) according to the manufacturer's instructions. Unconjugated iodine was removed using a Micro Bio-Spin 6 chromatography column (Bio-Rad, Hercules, Calif., United States of America). Cells (50 μl) were added to ¹²⁵I labeled CFH (1 μg protein in 50 μl) and the mixture was incubated at 22° C. for 30 minutes. For competitive binding, the cells were first incubated with 10 μg unlabeled CFH for 30 minutes and then ¹²⁵I labeled CFH was added. The cells were washed 3 times with PBS, and bound cpm was detected in a gamma counter (Packard). Each test was repeated in triplicate.

Preparation of serum IgG. Each pooled sample of 3 patients' sera (500 μl) was added to an equal volume 0.9% NaCl. Saturated ammonium sulfate (1000 μl) was added dropwise to this solution with constant mixing at 4° C. A precipitate was allowed to form over a period of 2 hours at 4° C. with continued mixing. The precipitate was collected by centrifugation for 15 min at 12,000×g, 4° C. The pellet was dissolved in cold PBS buffer. The IgG solution was dialyzed overnight at 4° C. against 4 liters of PBS. The concentration of precipitated IgG was calculated by Bradford protein assay (Bio-Rad). The final IgG concentration was adjusted to 6 mg/ml.

Deposition of C3-related fragments. We prepared Veronal-buffered saline (VBS), pH 7.4, with the addition of 0.1% (w/v) gelatin and 1 mM MgCl₂ (GVBS), as described previously (E. Wagner, H. Jiang, M. M. Frank, in Clinical Diagnosis and Management by Laboratory Methods J. B. Henry, Ed. (W. B. Saunders Company, Philadelphia, Pa., 2001) pp. 892-913), but omitting CaCl₂ to avoid the activation of the classical pathway. A549 and H661 cells were grown to 80% confluence in 75-cm² flasks and detached with trypsin-EDTA. Cells were collected and resuspended at a concentration of 2×10⁶/ml in GVBS. Patient IgG (1.5 mg/ml final concentration) and normal human serum (1:8 final dilution) were added to GVBS in a total volume of 100 μl. Parallel reactions contained only normal human serum in GVBS for data normalization. These mixtures were incubated at 4° C. for 30 minutes, added to cells (100 μl) and incubated at 37° C. for 30 minutes. To terminate the reactions, 1 ml ice cold GVBS was added, the mixtures were centrifuged and the supernatant was aspirated. As a background control, normal human serum was first incubated at 56° C. for 30 minutes to inactivate complement components. Goat anti-human C3 antibody (Complement Technology, Inc.) was labeled with ¹²⁵I using lodobeads as described above. Cells were washed with PBS and incubated with ¹²⁵I-labeled anti-C3 antibody at 22° C. for 30 minutes. The cells were washed 3 times with PBS, and bound cpm was detected in the gamma counter. Each test was repeated in triplicate. Data were collected, background (cpm with heated normal serum) was subtracted, and the resulting values were normalized with the cpm from unheated normal serum.

Immunohistochemistry. Following standard deparaffinization, immunohistochemistry was performed on individual sections (5-7 μm thick) using a goat anti-human CFH antiserum (Quidel, San Diego, Calif., United States of America). After blocking endogenous peroxidase activity and non-specific protein binding, sites with undiluted donkey serum for 10 min at room temperature, primary antibody was applied at 1:1000 dilution and incubated with the slides for 1 h at room temperature. Detection of immune complexes was with a goat-HRP polymer kit (BioCare Medical, Concord, Calif., United States of America), used according to the manufacturer's instructions. Bound antibody was detected with the chromogen 3,3′-diaminobenzidine and counterstaining was with Harris hematoxylin. Slides were dehydrated and cleared prior to coverslipping.

Statistical Analysis. A Fisher's exact test was used to calculate statistical significance.

### Example 5

Autoantibodies against alpha-glucosidase (GANAB) have also been discovered in some patients. These autoantibodies were identified in accordance with approaches described herein for Examples 1-4. These autoantibodies can also be used as therapeutic and/or diagnostic agents in accordance with the methods disclosed herein.

### Example 6

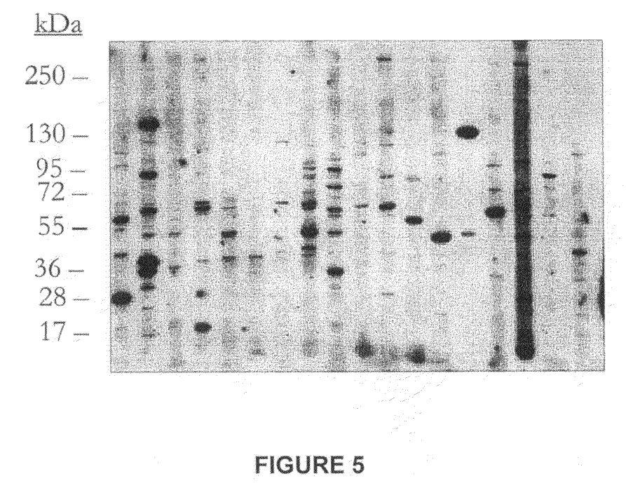

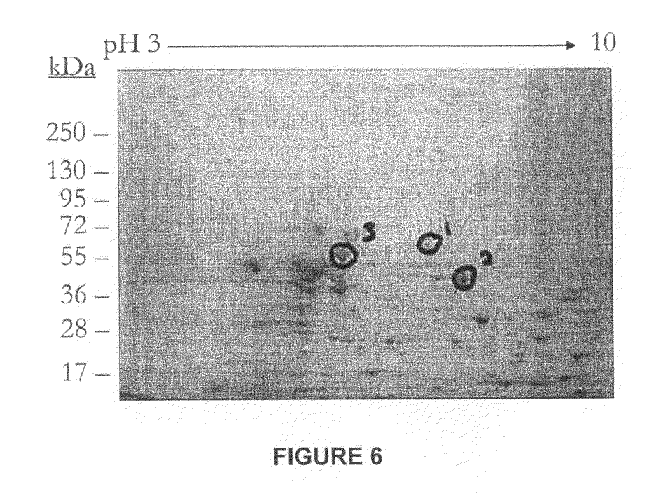

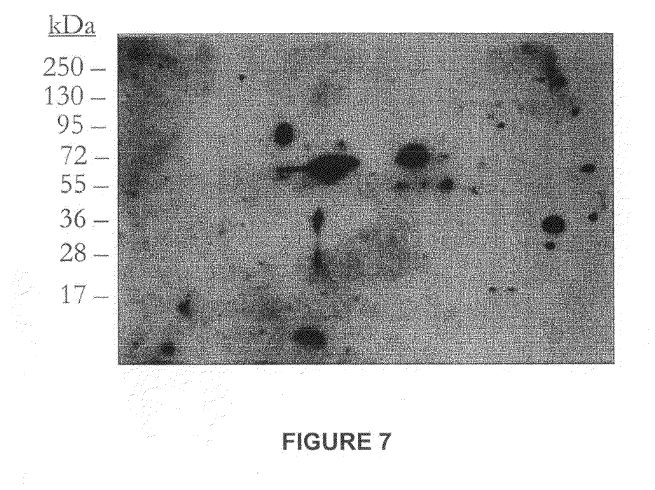

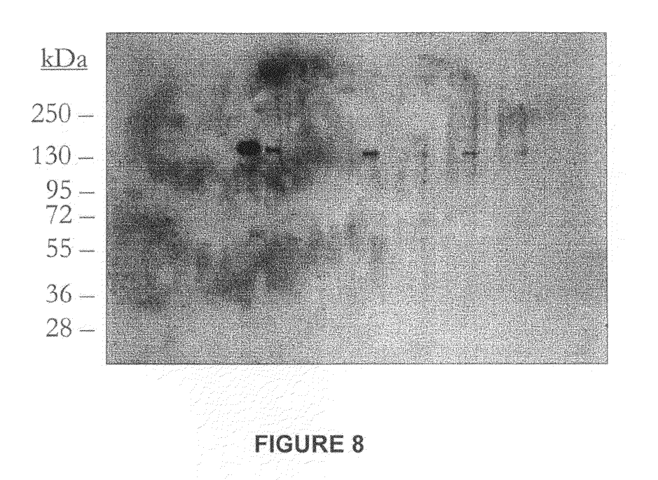

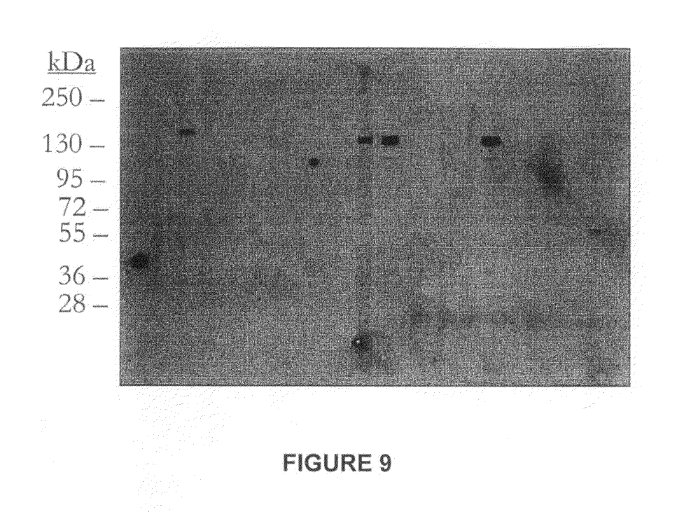

Materials and Methods. Serum samples were collected from patients with diagnosed non-small cell lung cancer. A Western blot (FIG. 5) was performed using a pool of lysates from three lung adenocarcinoma cell lines. The blot was then probed using sera from these patients. Samples containing many discrete bands (corresponding to complexes of proteins and autoantibodies) were further analyzed utilizing 2D-PAGE and Western blotting (FIGS. 6 and 7). Signals produced when the lysate proteins were probed with patient serum were then lined up with stained gels. Corresponding spots on the gel were excised and sent to the Duke Proteomics Core Facility (Duke University, Durham, N.C., United States of America) for digestion of the proteins and analysis of the resulting peptides by nanoscale liquid chromatography-tandem mass spectrometry. Upon identification, purified proteins were purchased. Next, the presence of autoantibodies to these proteins, as well as Complement Factor H (CFH) and α-glucosidase, were determined using the sera of twenty patients with NSCLC and twenty matched controls using Western blotting (FIGS. 8 and 9).

TABLE 1
Characteristics of NSCLC patient cohort and matched controls.

| Characteristic | Cancer (n = 20) | Control (n = 20) |
|---|---|---|
| Age (years) | 60.25 ± 9.14 | 60.25 ± 9.22 |
| Sex | | |
| Male | 10 | 10 |
| Female | 10 | 10 |
| Smoking History (pack years) | 54.33 ± 28.27 | 54.30 ± 29.38 |

TABLE 2
Proteomics Results for Spot #1.

| | Swiss Prot | Molecular | | | Protein |
|---|---|---|---|---|---|
| | Accession | Weight | | Peptides | coverage |
| Protein name | number | (kDa) | pI | matched | (%) |
| Stress-induced- | P31948 | 62.6 | 6.40 | 40 | 55 |
| phosphoprotein 1 | | | | | |
| Bifunctional purine | P31939 | 64.6 | 6.27 | 15 | 29 |
| biosynthesis | | | | | |
| protein PURH | | | | | |
| Heat shock protein | P10809 | 61.0 | 5.70 | 5 | 12 |
| 60 | | | | | |
| UDP-glucose 6- | O60701 | 55.0 | 6.73 | 5 | 10 |
| dehydrogenase | | | | | |
| D-3- | O43175 | 56.7 | 6.29 | 4 | 8 |
| phosphoglycerate | | | | | |
| dehydrogenase | | | | | |

TABLE 3
Number of Patients with Autoantibodies for Each Target Protein.

| Target protein | NSCLC patients | Controls |
|---|---|---|
| 14-3-3^{ε} | 4/20 | 4/20 |
| HSP-60 | 6/20 | 9/20 |
| STI1 | 12/20 | 11/20 |
| α-glucosidase | 3/20 | 3/20 |
| CFH | 6/20 | 4/20 |

Discussion. Proteins identified from spots excised from the 2D-PAGE and Western blotting included stress-induced-phosphoprotein 1 (STI1), α-enolase, HSP-60, and 14-3-3ε. The number of NSCLC patients having autoantibodies directed at these proteins did not appear to differ significantly from controls. There are many possible explanations for this finding. First of all, the size of this sample is too small to generalize this conclusion to the entire population of NSCLC patients. Also, determining the concentration of protein to be used in the assay is difficult. While increasing the concentration can enable the detection of an autoantibody present in the serum in a low titer, it can also introduce the possibility of nonspecific binding of other autoantibodies. In addition, some patients could have autoantibodies that are directed at areas of the protein that are mutated or alternatively spliced. Such samples would not recognize the purified protein and be read as false negatives on this assay. But, this approach has determined a successful protocol for identifying targets of autoantibodies in NSCLC patients and has already identified four such proteins. These proteins can appear on a microarray in order to test the autoantibody status of a much larger number of NSCLC patients.

### Example 7

Autoantibodies against the entities listed in Tables 2-4 have also been discovered in some patients. These autoantibodies were identified in accordance with approaches described herein for Examples 1-6. These autoantibodies can also be used as therapeutic and/or diagnostic agents in accordance with the methods disclosed herein.

TABLE 4

| | | Uniprot ID |
|---|---|---|
| | | (Swiss Prot |
| | Gene | Accession |
| Protein Name | Name | number) |
| alpha-enolase | ENO1 | P06733 |
| stress-induced-phosphoprotein 1 | STIP1 | P31948 |
| Hypoxia up-regulated 1 | HYOU1 | Q9Y4L1 |
| 14-3-3 epsilon | YWHAE | P62258 |
| HSP60 | HSPD1 | P10809 |
| GRP78 | HSPA5 | P11021 |
| complement factor H | CFH | P08603 |
| neutral alpha-glucosidase AB | GANAB | Q14697 |
| Vimentin | VIM | P08670 |
| peroxiredoxin-1 | PRDX1 | Q06830 |
| elongation factor 1-alpha | EEF1A1 | P68104 |
| cyclophylin A | PPIA | P62937 |
| Ran GTPase | RAN | P62826 |
| HSP70 | HSPA1A | P08107 |
| protein disulfide-isomerase A3 | PDIA3 | P30101 |
| Thioredoxin | TXN | P10599 |
| transketolase | TKT | Q16832 |
| retinal dehydrogenase | ALDH1A1 | P00352 |
| Stathmin 1 | STMN1 | P16949 |
| Nucleolin | NCL | P19338 |
| elongation factor Tu, mitochondrial | TUFM | P49411 |
| p53 | TP53 | P04637 |
| 14-3-3theta | YWHAQ | P27348 |
| PGP9.5 | UCHL1 | P09936 |
| annexin 1 | ANXA1 | P04083 |
| protein kinase a catalytic subunit | PRKACA | P17612 |

### References

All references listed herein, including patents, patent applications, data base citations (including but not limited to Swiss Prot Accession

Numbers) and scientific literature, are incorporated herein by reference in their entireties to the extent that they supplement, explain, provide a background for, or teach methodology, techniques, and/or compositions employed herein.
1. O. J. Finn, J Immunol 178, 2615 (Mar. 1, 2007).
2. S. Rodriguez de Cordoba, J. Esparza-Gordillo, E. Goicoechea de Jorge, M. Lopez-Trascasa, P. Sanchez-Corral, Mol Immunol 41, 355 (June 2004).
3. D. Ajona et al., Cancer Res 64, 6310 (Sep. 1, 2004).
4. D. Ajona, Y. F. Hsu, L. Corrales, L. M. Montuenga, R. Pio, J Immunol 178, 5991 (May 1, 2007).
5. E. Wilczek et al., Int J Cancer 122, 2030 (May 1, 2008).
6. S. Junnikkala et al., Br J Cancer 87, 1119 (Nov. 4, 2002).
7. Z. Z. Cheng et al., Clin Chem 51, 856 (May, 2005).
8. P. Gasque, A. Thomas, M. Fontaine, B. P. Morgan, J Neuroimmunol 66, 29 (May, 1996).
9. S. Junnikkala et al., J Immunol 164, 6075 (Jun 1, 2000).
10. K. Jurianz et al., Mol Immunol 36, 929 (September-October 1999).
11. K. M. Murphy, P. Travers, M. Walport, Janeway's Immunobiology (Garland Science, London, England, ed. 7th, 2007), pp.
12. M. A. Dragon-Durey et al., J Am Soc Nephrol 16, 555 (February 2005).
13. E. Gumus et al., Int J Urol 11, 1070 (December 2004).
14. P. J. Mintz et al., Nat Biotechnol 21, 57 (January 2003).
15. S. Varsano, L. Rashkovsky, H. Shapiro, D. Ophir, T. Mark-Bentankur, Clin Exp Immunol 113, 173 (August 1998).
16. R. Sayegh, J. T. Awwad, C. Maxwell, B. Lessey, K. Isaacson, J Clin Endocrinol Metab 80, 1021 (March 1995).
17. E. Wagner, H. Jiang, M. M. Frank, in Clinical Diagnosis and Management by Laboratory Methods J. B. Henry, Ed. (W. B. Saunders Company, Philadelphia, Pa., 2001) pp. 892-913.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for detecting cancer in a subject, the method comprising:
detecting the presence and/or amount of a cancer-associated autoantibody in a sample from the subject.

2. A method for managing treatment of a subject with potential cancer, the method comprising:
detecting the presence and/or amount of a cancer-associated autoantibody in a sample from the subject; and
managing the treatment of the subject with potential cancer based on the presence or amount of the cancer-associated autoantibody.

3. A method for molecular staging of a tumor or suspected tumor, the method comprising:
detecting the presence and/or amount of a cancer-associated autoantibody in a sample from the subject; and
determining the molecular stage of the tumor or suspected tumor based on the presence or amount of the cancer-associated autoantibody.

4. A method for assigning a subject to a high-risk group for cancer, the method comprising:
detecting the presence and/or amount of a cancer-associated autoantibody in a sample from the subject; and
assigning the subject to a group having a high-risk of cancer based on the presence or amount of the cancer-associated autoantibody.

5. The method of any one of claims 1-4, wherein the sample is a serum sample or a plasma sample.

6. The method of any one of claims 1-5, wherein the subject is a human subject.

7. The method of any one of claims 1-6, where in' the cancer is lung cancer.

8. The method of any one of claims 1-7, comprising detecting an autoantibody against Complement Factor H (CFH), an autoantibody against alpha-glucosidase (GANAB), an autoantibody against STIP1 (Stress-induced-phosphoprotein 1), an autoantibody against alpha-enolase, an autoantibody against 14-3-3 (14-3-3 protein epsilon), or an autoantibody against HSP 60 (60 kDa heat shock protein), or detecting any combination of the aforementioned autoantibodies.

9. The method of any one of claims 1-7, comprising detecting an autoantibody against one or more of the entities listed in Table 4, or detecting any combination thereof.

10. A kit for detecting the presence and/or amount of a cancer-associated autoantibody in a sample from the subject, the kit comprising:
a binding partner specific for a cancer-associated auto-antibody; and
directions for detecting the presence of and/or measuring the amount of a cancer-associated autoantibody in a sample from the subject.

11. The kit of claim 10, comprising a binding partner specific for an autoantibody against Complement Factor H (CFH), a binding partner specific for an autoantibody against alpha-glucosidase (GANAB), a binding partner specific for an autoantibody against STIP1 (Stress-induced-phosphoprotein 1), a binding partner specific for an autoantibody against alpha-enolase, a binding partner specific for an autoantibody against 14-3-3 (14-3-3 protein epsilon), or a binding partner specific for an autoantibody against HSP 60 (60 kDa heat shock protein), or any combination of the aforementioned binding partners.

12. The kit of claim 10, comprising a binding partner specific for an autoantibody against one or more of the entities listed in Table 4, or any combination thereof.

13. The kit of claim 10, wherein the binding partner is conjugated to a solid support, and comprising a second specific binding partner for the autoantibody.

14. The kit of claim 13, wherein the second specific binding partner is an antibody.

15. The kit of claim 12 or 13, wherein the second specific binding partner is conjugated to a detectable group.

16. The kit of claim 15, wherein the detectable group is selected from the group consisting of radioactive labels, fluorescent labels, enzyme labels and fluorescent labels.

17. The kit of claim 10, comprising one or more of buffering agents, protein stabilizing agents, enzyme substrates, background reducing agents, control reagents, an apparatus for conducting the detection, and any necessary software for analysis and presentation of results.

18. A method of treating cancer in a subject, the method comprising administering to a subject having a cancer an effective amount of an antibody, which can optionally be a bispecific antibody, having the immunoreaction characteristics of a cancer-associated autoantibody; an antigen for a cancer-associated autoantibody; or both an antibody having the immunoreaction characteristics of a cancer-associated autoantibody and an antigen for a cancer-associated autoantibody.

19. The method of claim 18, where in the cancer is lung cancer.

20. The method of claim 18 or claim 19, comprising administering an antibody against Complement Factor H (CFH), an antibody against alpha-glucosidase (GANAB), an antibody against STIP1 (Stress-induced-phosphoprotein 1), an antibody against alpha-enolase, an antibody against 14-3-3 (14-3-3 protein epsilon), or an antibody against HSP 60 (60 kDa heat shock protein), or any combination of the aforementioned antibodies.

21. The method of claim 18 or claim 19, comprising administering an antibody against one or more of the entities listed in Table 4, or administering any combination thereof.

22. The method of claim 18 or claim 19, comprising administering a Complement Factor H (CFH) antigen, an alpha-glucosidase (GANAB) antigen, a STIP1 (Stress-induced-phosphoprotein 1) antigen, an alpha-enolase antigen, a 14-3-3 (14-3-3 protein epsilon) antigen, or a HSP 60 (60 kDa heat shock protein) antigen, or any combination of the aforementioned antigens.

23. The method of claim 18 or claim 19, comprising administering an antigen prepared from one or more of the entities listed in Table 4, or administering any combination thereof.

24. The method of any one of claims 18-23, comprising administering an adjuvant to the subject.

25. A composition for treating cancer in a subject, the composition comprising an effective amount of an antibody, which can optionally be a bispecific antibody, having the immunoreaction characteristics of a cancer-associated autoantibody; an antigen for a cancer-associated autoantibody; or both an antibody having the immunoreaction characteristics of a cancer-associated autoantibody and an antigen for a cancer-associated autoantibody; and a pharmaceutically acceptable carrier.

26. The composition of claim 25, where in the cancer is lung cancer.

27. The composition of claim 25 or claim 26, comprising an antibody against Complement Factor H (CFH), an antibody against alpha-glucosidase (GANAB), an antibody against STIP1 (Stress-induced-phosphoprotein 1), an antibody against alpha-enolase, an antibody against 14-3-3 (14-3-3 protein epsilon), or an antibody against HSP 60 (60 kDa heat shock protein), or any combination of the aforementioned antibodies.

28. The composition of claim 25 or claim 26, comprising an antibody against one or more of the entities listed in Table 4, or any combination thereof.

29. The composition of claim 25 or claim 26, comprising a Complement Factor H (CFH) antigen, an alpha-glucosidase (GANAB) antigen, a STIP1 (Stress-induced-phosphoprotein 1) antigen, an alpha-enolase antigen, a 14-3-3 (14-3-3 protein epsilon) antigen, or a HSP 60 (60 kDa heat shock protein) antigen, or any combination of the aforementioned antigens.

30. The composition of claim 25 or claim 26, comprising an antigen prepared from one or more of the entities listed in Table 4, or any combination thereof.

31. The composition of any one of claims 25-30, comprising an adjuvant.

32. An isolated and purified antibody having the immunoreaction characteristics of an autoantibody against Complement Factor H (CFH).

33. An isolated and purified antibody having the immunoreaction characteristics of an autoantibody against alpha-glucosidase (GANAB).

34. An isolated and purified antibody having the immunoreaction characteristics of an autoantibody against STIP1 (Stress-induced-phosphoprotein 1).

35. An isolated and purified antibody having the immunoreaction characteristics of an autoantibody against alpha-enolase.

36. An isolated and purified antibody having the immunoreaction characteristics of an autoantibody against 14-3-3 (14-3-3 protein epsilon).

37. An isolated and purified antibody having the immunoreaction characteristics of an autoantibody against HSP 60 (60 kDa heat shock protein).

38. An isolated and purified antibody having the immunoreaction characteristics of an autoantibody against one or more of the entities listed in Table 4.
